(19) United States
(12) Patent Application Publication
(10) Pub. No.: US 2008/0187995 A1
(43) Pub. Date: Aug. 7, 2008

(54) USE OF TOPOGRAPHIC CUES TO MODULATE STEM CELL BEHAVIORS

(76) Inventors: Christopher J. Murphy, Madison, WI (US); Paul F. Nealey, Madison, WI (US); Daniel R. McFarlin, Madison, WI (US); Sara J. Liliensiek, Madison, WI (US); George A. McKie, Madison, WI (US)

Correspondence Address: BRINKS HOFER GILSON & LIONE, P.O. BOX 10395, CHICAGO, IL, 60610, US

(21) Appl. No.: 11/860,886

(22) Filed: Sep. 25, 2007

Related U.S. Application Data
(60) Provisional application No. 60/848,209, filed on Sep. 29, 2006.

(51) Int. Cl. C12N 5/00; C12N 5/02

(52) U.S. Cl. 435/377; 435/395

(57) ABSTRACT

Surfaces, kits, and methods for the modulation of cell behavior in vitro by patterned nanoscale topography. The invention is particularly useful for providing means to affect and control the growth and differentiation of human embryonic stem cells.

### CROSS-REFERENCE TO RELATED APPLICATIONS

This invention claims priority to U.S. Provisional Patent Application Ser. No. 60/851,662, filed Sep. 29, 2006.

### FIELD OF THE INVENTION

The present invention relates generally to the field of cell growth and culture. More particularly, the present invention provides novel topographic substrates and methods for controlling the growth and differentiation of cells in vitro.

### BACKGROUND

In the vertebrate body, basement membranes and the extracellular matrix serve as the substrate upon which overlying cellular structures grow. There are physical and chemical differences in the surfaces of divergent basement membranes that can exert influence on the cells (Whitesides et al., 2005, Sci. Prog. 88: 17-48). Research has shown that substrate topography influences cells in a manner distinct from surface chemistry. One physical difference in the topography of divergent basement membranes is the size of pores and ridges. In vivo, cells never see flat surfaces: on the nanoscale, no basement membrane or extracellular matrix is flat.

The great majority of features in the extracellular environment are in the submicron to nanoscale range, ensuring that an individual cell is in contact with numerous topographic features. Topographic features have been shown to affect a wide range of cellular behaviors (Abrams et al., 2002, Cells Tissues Organs 170: 251-257; Curtis and Wilkinson, 1997, Biomaterials 18:1573-1583). Surface features with dimensions of tens to hundreds of nanometers have been reported to affect proliferation, alignment, adhesion, migration, growth factor sensitivity, and cell viability (Clark et al., 1991, J. Cell Sci. 99: 73-77; Dalby et al. 2002, Tissue Eng. 8: 1099-1108; Fan et al., 2002, J. Neurosci. Methods, 120: 17-23; Teixeira et al., 2003, J. Cell Sci. 15: 1881-1892; Chung et al., 2003, Biomaterials 24: 4655-4661; Washburn et al., 2004, Biomaterials 25: 1215-1224; Karuri et al., 2004, J. Cell Sci. 117: 3153-3164). In many cases, the impact of topographic cues is manifest only when the size scale of the features is in the submicron to nanoscale range. Some findings point to shifts in cell behaviors as substratum features decrease from micron scale to nanoscales characteristic of those found in the native environment.

It has been shown that topography can exert influence on mesenchymal stem cells. For example, Castano et al. (2004, Macromol. Biosci. 4: 785-794) focused on the thickness of polypyrrole films and their potential as a biocompatible material for rat mesenchymal stem cells. Others have investigated the potential of electrospun porous scaffolds of randomly oriented 500 nm to 900 nm diameter nanofibers for cartilage repair (Li et al., 2005, Biomaterials, 26: 599-609; Shin et al., 2004, Tissue Eng. 10: 33-41). U.S. Pat. No. 6,942,873 discloses microfabrication of membranes for growing cells. These works were concerned with the biocompatibility of the material and did not investigate the effects of varying nanofiber size.

Human embryonic stem (HES) cells are capable of differentiating into cells types normally derived from all three germ layers of early human development. The unique ability of HES cells to form tissue of ectodermal, mesodermal and endodermal origin is part of the impetus for the rapidly growing field of stem cell engineering. Another driving force behind HES cell research is their unique potential for unlimited self-renewal. Unlike stem cells derived from adult tissues, which have a limited number of cell doublings, HES cells cultured under the right conditions have the potential to divide indefinitely, without losing their pluripotent properties. However, spontaneously differentiated colonies must be removed regularly to maintain pluripotent cultures. Characterizing the environmental factors and mechanisms that can influence HES cell differentiation and self-renewal will greatly improve our understanding of human development, disease, and aging, while also increasing the potential of utilizing HES cells for treatment of human ailments.

In studies involving cell behavior in vitro, and particularly in studies involving growth and differentiation of embryonic stem cells in vitro, it is important to control cellular behavior including growth, differentiation, morphology, alignment, adhesion, proliferation, and migration. In the future, controlled growth and differentiation of stem cells into particular cell types and tissues may be used to replace or help repair damaged cells that result from injury, disease, and aging. The fundamental challenge of current stem cell research is characterization of environmental factors that modulate differentiation and self-renewal. The present invention provides topographic substrates and methods for facilitating this objective.

### BRIEF SUMMARY

This invention provides methods for growing cells in vitro which include contacting a suspension of cells in a cell culture medium with a patterned surface for growth of cells. The patterned surface includes a planar surface having nanotextured topography with longitudinal grooves and projections extending perpendicularly to the planar surface. The methods include incubating the cells under cell growth or differentiation conditions to maintain the cells in their desired growth and differentiation state. The cells may be stem cells, preferably embryonic stem cells, and more preferably human embryonic stem cells.

The methods may be practiced with patterned surfaces having a projection pitch size of between about 40 nm and about 8000 nm. The size ratio of a projection to a longitudinal groove may be approximately 1:1.

The methods may include growing cells such that, under culture conditions that promote cell differentiation, the topography of the surface promotes cell differentiation as compared to cell differentiation on a surface in the absence of the topography.

The methods may include growing cells such that, under culture conditions that promote cell self-renewal, the topography of the surface promotes cell self-renewal as compared to cell self-renewal on a surface in the absence of the topography.

The methods may be practiced with patterned surfaces having a planar surface formed from a material with functional groups capable of forming a covalent bond with a biomolecule selected from the group consisting of a peptide, a protein, a polynucleotide, a polysaccharide, a lipid, a growth factor, and a bioactive agent.

This invention provides a kit which includes a patterned surface for growth of cells including a planar surface having nanotextured topography with longitudinal grooves and projections extending perpendicularly to the planar surface, and an undifferentiated cell. The undifferentiated cell may be a stem cell, preferably, an embryonic stem cell, and more preferably a human embryonic stem cell.

The patterned surface in the kit may include a planar surface with projections having a projection pitch size of between about 40 nm and about 8000 nm. The size ratio of a projection to a longitudinal groove may be approximately 1:1.

The kit may further include culture medium.

The kit may provide that, under culture conditions that promote cell differentiation, the topography of the surface promotes cell differentiation as compared to cell differentiation on a surface in the absence of the topography.

The kit may provide that, under culture conditions that promote cell self-renewal, the topography of the surface promotes cell self-renewal as compared to cell self-renewal on a surface in the absence of the topography.

The patterned surface in the kit may include a planar surface that is formed from a material with functional groups capable of forming a covalent bond with a biomolecule selected from the group consisting of a peptide, a protein, a polynucleotide, a polysaccharide, a lipid, a growth factor, and a bioactive agent.

### BRIEF DESCRIPTION OF THE DRAWINGS

FIG. 1 is a schematic diagram of the major steps involved in a method for producing the surfaces of the present invention.

FIG. 2 shows images of molded patterned surfaces.

FIG. 3 is a graph of the values for the Young's modulus (kPa) as a function of the mol % of the cross-linker.

FIG. 4 is an (A) image of a micrograph showing cysteine containing peptides immobilized onto nanoscale topography, and (B) schematic diagram of the functionalization process.

FIG. 5 is an image of a micrograph showing a HES cell colony at the intersection between a flat surface (to the left of the vertical line) and a topographically patterned surface (to the right of the vertical line).

FIG. 6 shows images of micrographs of human embryonic stem cells that have migrated away from their colonies on surfaces with different pitch (1200-4000 nm).

FIG. 7 depicts images of fluorescence micrographs depicting proliferation rates of HES cells on flat (A) and patterned (B-D) surfaces.

FIG. 8 is a graph showing how nanoscale topography reduces the frequency of spontaneous differentiation in HES cell cultures.

FIG. 9 is an image showing stem cell proliferation.

FIG. 10 is an image showing stem cell self-renewal.

### DETAILED DESCRIPTION OF THE PRESENTLY PREFERRED EMBODIMENTS

The following definitions are provided in order to aid the reader in understanding the detailed description of the present invention.

“A”, “an”, “the” and the like, unless otherwise indicated, include plural forms.

“Nanotexture” is texture on a nanometer (10⁻⁹ m) scale. For the purposes of this invention, nanotexture refers to texture in the range of about 40 nm to about 8000 nm.

“Pitch” refers to the width that consists of the ridge width plus the groove width.

“Young's modulus” or “elastic modulus”, “modulus of elasticity” is a measure of the stiffness of a given material. It is the ratio of the rate of change of stress with strain. Stiffness is the resistance of an elastic body to deflection by an applied force.

“Compliance” is the reciprocal (inverse) of stiffness (elastance). Compliance of the surface is the reciprocal (inverse) value of the stiffness of the surface.

“Cell behavior” refers to anything that a cell does in response to its environment. For cells grown in vitro, it refers to anything that cells do in response to the culture conditions, media, environment, endogenous or exogenous stimuli, etc. Cell behavior may involve action and response to stimulation. In particular, cell behavior for the purposes of this invention includes growth, differentiation, morphology, alignment, adhesion, proliferation, migration, growth factor sensitivity, and cell viability.

“Functionalization” of a surface refers to the attachment of a functional moiety to the surface. The attachment can, for example, include conjugating a biologically active molecule or multiple molecules to the surface.

This invention provides compositions and methods for the modulation of the behavior of cells grown in vitro. Using the values for the compliance of the surface, it is possible to design patterned structures that affect the behavior of cells grown in vitro. In one aspect, the invention provides methods that use topographic cueing by designing substratum topography to affect and control cell behavior.

This invention provides a patterned surface for the growth of cells, which include a planar surface having nanotextured topography including longitudinal grooves in the planar surface and projections extending perpendicularly to the planar surface. The nanotextured topography provides enhanced means to affect growth and development of the cells as compared to the growth and development of the cells on a surface in the absence of the nanotextured topography.

The patterned surface may have nanotextured topography that includes projections having a projection pitch size of between about 10 nm and about 10000 nm, and preferably a projection pitch size of between about 40 nm and about 8000 nm. The size ratio of a projection to a longitudinal groove may be approximately 1:1.

The patterned surface may, under culture conditions that promote cell differentiation, promote cell differentiation as compared to cell differentiation on a surface in the absence of the topography.

The patterned surface may, under culture conditions that promote cell self-renewal, promote cell self-renewal as compared to cell self-renewal on a surface in the absence of the topography.

The patterned surface may include an exposed functional group being capable of forming a covalent bond with a molecule. The molecule may be selected from the group consisting of a peptide, protein, polynucleotide, polysaccharide, growth factor, and a bioactive agent.

The introduction of topography as a modulator of cell behavior in vitro increases predictability of cell behavior outcomes. The substrate topography can be used to guide cell behavior including cell growth, differentiation, development, and proliferation. The ability to spatially localize and control interactions of cell types on polymeric materials presents an opportunity to engineer hierarchically and more physiologically correct tissue analogs for mechanical, biochemical, functional, experimental, and clinical purposes.

The use of topographic cueing using specific dimensional features may participate in determining the differentiation fate of embryonic stem cells and participate with other soluble factors in ultimately determining the pathways of differentiation pursued (or the retention of the dedfferentiated state). The physical topography of the substrates can be varied and used to modulate the growth and developmental state of stem cells as well as other undifferentiated cells, including ocular cells, PC 12 cells, fibroblasts, etc.

In one aspect, the physical topography of the substrates upon which human embryonic stem (HES) cells are cultured can influence the frequency of their spontaneous differentiation and self-renewal. For example, growing HES cells on surfaces with ridges spaced about 400 nm apart promotes maintenance of the pluripotent state of HES cells. Conversely, growing HES cells on surfaces with ridges spaced about 1200 nm or more promotes differentiation.

HES cell self-renewal makes it possible to grow the large number of cells necessary for effective treatment of human ailments, from a limited number of starting cells. Conversely, HES cells grown on topographic substrates under conditions that lack self-renewal promoters (e.g. without MEF—Mouse Embryonic Fibroblasts) feeder cells) are more prone to the loss of stem cell markers, i.e. are more prone to differentiation.

This invention has broad application to areas such as in vitro cell culture and stem cell biology. It is thus possible to consider nanoscale topographic substrates and cues as a fundamental factor for the predictable culture of embryonic stem cells in the lab and in medical implants.

### Fabrication of Patterned Surfaces

This invention contemplates the design and manufacturing of a variety of patterned surfaces for culturing cells. Nonlimiting examples of materials that can be produced with patterned surfaces include consumables such as microplates, culture dishes, microscope slides, chips, etc.

The invention provides methods for producing a patterned surface by using a variety of fabrication methods, exemplified by but not limited to soft lithographic techniques, electroless deposition of gold onto porous membranes with subsequent digestion of membrane, scintering, use of electrospun membranes (e.g. Donaldson Co. Minneapolis, Minn.), use of block copolymers and abrasive spraying (e.g. sandblasting).

Surfaces can be made using different materials. Surfaces can be fabricated using, for example, metals, alloys, polymer, silicon, and mixtures thereof.

In some embodiments, surfaces are pretreated prior to patterning. Pretreatment can, for example, be used to smoothen the surface.

The techniques of microfabrication and micromachining have been recently used to create precisely controlled biomaterial surfaces via photopatterning and etching, e.g. as described in Desai et al., 1998, Biotechnol. Bioeng. 57: 118-120; Bhatia et al., 1998, Biotech. Prog. 14: 378-387; Chen et al., 1998, Biotech. Prog. 14: 356-363, all of which are incorporated herein by reference. Production of nanograting with soft lithography has been described by Yim et al., 2007, Exp. Cell Research 313: 1820-1829), which is incorporated herein by reference. Micro- and nano-fabricated substrates can provide unique advantages over traditional biomaterials due to their ability to control surface micro- and nano-architecture, topography, and feature size in the nanometer and micron size scale, and control surface chemistry in a precise manner through biochemical coupling or photopatterning processes.

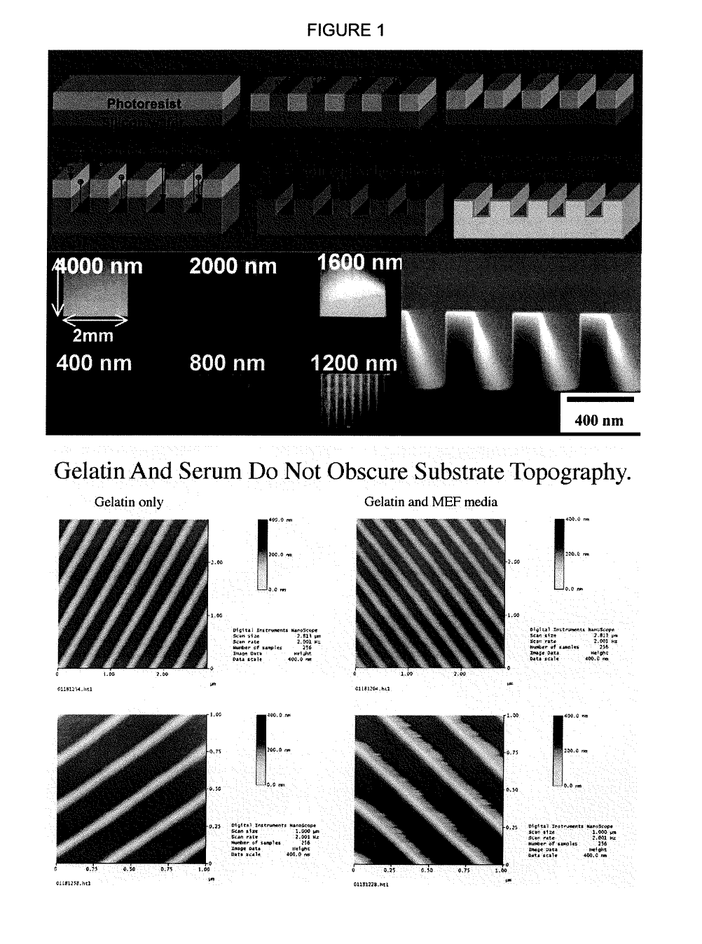

The major steps involved in a preferred method of fabricating patterned surfaces according to this invention are schematically outlined in FIG. 1 and are also described in the examples section below. In one embodiment, soft lithography is used to produce patterned surfaces with desired compliance of the surface. However, the actual method of fabricating patterned surfaces can vary, depending, e.g., on the material used, the application desired (e.g. differentiation or self-renewal), and the cell type that will be grown in culture using the topographic surface.

It is not intended that the patterned surface having nanotextured topography of the present invention be limited to a particular dimension. Generally speaking, the compliance of the surface will be the factor determining the desired topography for a particular application (e.g. desired cell growth, desired cell self-renewal, desired cell differentiation, etc.). In one preferred embodiment, the nanotextured topography has projections with a pitch size of between about 40 nm and about 8000 nm. More preferably, the pitch size of the projections is between about 400 nm and about 1400 nm.

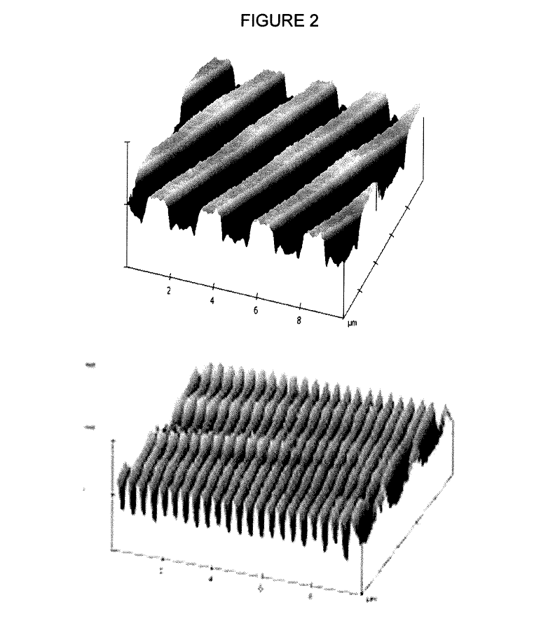

Exemplary images of molded patterned surfaces of UV curable hydrogels are shown in FIG. 2. PolyHEMA coated culture substrates with submicron to nanoscale groove and ridge topography are shown. The compliance of the surfaces can be tailored by changing cross-link density.

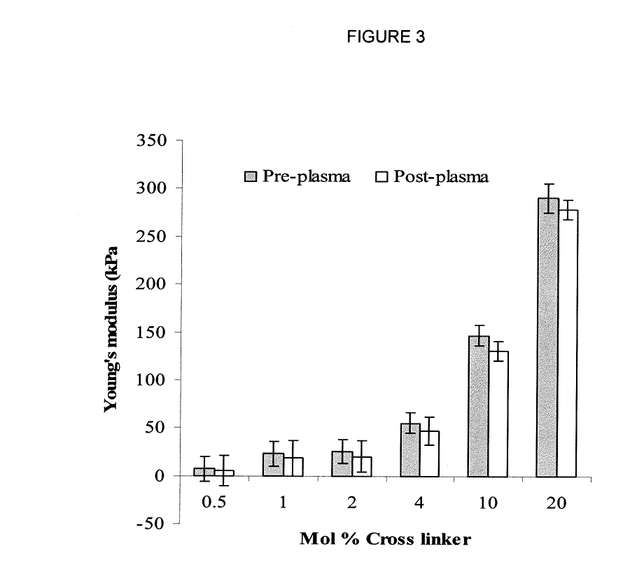

FIG. 3 shows the values for the Young's modulus (kPa) as a function of the mol % of the cross-linker.

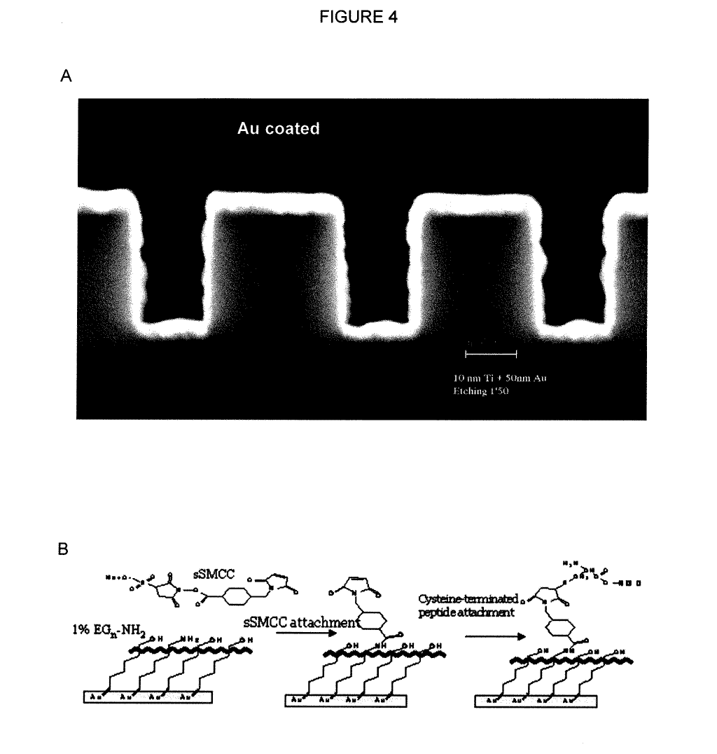

In some embodiments, the surface chemistry of the nanotextured surfaces can be altered. For example, chemical bonding protocols which alter the surface chemistry of nanotextured silicone and other substrata can be used to functionalize the desired surface in order to modulate cell attachment, growth, and differentiation. Functionalized surfaces could include attached molecules such as peptides, proteins, polynucleotides, polysaccharides, lipids, growth factors, and other bioactive agents. Covalent attachment of peptides to the silicone surfaces can be used, as depicted schematically in FIG. 4.

Various protocols known in the art can be used for functionalization of the surfaces. The protocols will vary according to the type of molecule that is being attached to the surface and the surface material used. Many chemical methods, including the condensation of amines with activated carboxylic acids or with aldehydes, are convenient and applicable to most molecules (Wilbur et al., 1997; Horton et al., 1997). Others, including the coordination of Ni(II) complexes with oligo(histidine) motifs, have excellent selectivity (Sigal et al., 1996, Anal. Chem. 68: 490-497). PCT publication WO 98/30575, incorporated herein by reference, describes a method for conjugating macromolecules to other molecular entities using cycloaddition reactions such as the Diels-Alder reaction. For metal surfaces, alkylthiol chemistry is well known. Reductive amination can be used as a conjugation method for functionalization of proteins, e.g. as described in Wong, 1991, Chemistry of Protein Conjugation and Cross-Linking, CRC Press, Boston, which is incorporated herein by reference. Similarly, the method disclosed in U.S. Patent Publication No. 2006/0014003 A1, incorporated herein by reference, could be used for covalently binding molecules to the surfaces of this invention. While modification with a thiol group is shown for illustrative purposes in the examples section, one skilled in the art can modify that thiol group fairly easily to add linkers such as biotin, quinone, etc., that attach to biologically active molecules such as proteins, DNA, etc.

Functionalization of the surfaces can be performed pre- and post-patterning. Functionalization that is performed pre-patterning of the surfaces will give grooves without attachment points. Functionalization that is performed post-patterning will give grooves without attachment points.

### Cell Behavior on Patterned Surfaces

Cell culturing on the patterned surfaces can be done according to standard procedures known in the art. For example, stem cells can be grown on the surfaces using culture medium obtained from WiCell (WiCell Research Institute, Madison, Wis.). One skilled in the art will know that different types of culture media can be used for different applications. For example, if the objective is to promote cell differentiation, the culture medium can be modified to contain compounds that promote differentiation; if the objective is to promote cell self-renewal, the culture medium can be modified to contain compounds that promote cell self-renewal. The significance of nanotopography in directing differentiation of adult stem cells was recently recognized (Yim et al., 2007, Exp. Cell Research 313: 1820-1829), which is incorporated herein by reference.

Other parameters that influence cell culture in vitro can also be used to induce or promote cell behavior as desired. These include environmental parameters such as temperature, pressure, etc.

It is not intended that the present invention be limited for culturing particular type of cells (or merely one cell type on surface). A variety of cell types (including mixtures of different cells) are contemplated. Indeed, any type of cell grown in vitro can be grown on the patterned surfaces. The cells could be undifferentiated cells from any mammal. In one embodiment, the cells are stem cells, preferably embryonic stem cells, and more preferably human embryonic stem cells. In another embodiment, the cells secrete a medically useful compound (e.g., hormone, cytokine, etc.). Such cells may be (but need not be) cells that have been manipulated by recombinant means to secrete such compounds.

Assaying of the cell behavior is performed using standard assays that measure cell behavior, such as cell growth, proliferation, migration, self-renewal, etc. Some of these techniques are described in the examples section below. Generally, what is important is a direct comparison of the cell behavior when cells are cultured on the patterned surface of this invention with the behavior of cells that are cultured on a flat surface.

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

### EXAMPLES

### Example 1

### Stem Cell Culture

H1 HES cells were maintained following the traditional WiCell protocol (WiCell Research Institute, Madison, Wis.), with minor alterations to the “pick to remove” method. In brief, tissue culture polystyrene was first coated with gelatin (0.1% solution in water). CF-1 strain mouse embryo fibroblasts were irradiated with about 8500-10000 cGray from cesium 137 before seeding as a feeder layer. HES cells were passaged onto fresh MEF feeder layers every 7 to 12 days and were fed daily at least six days a week. Collaginase treatment was used to harvest cells for passage after removing the differentiated portions of colonies. Areas of HES cell colonies that had differentiated were defined by morphological and non-refractive characteristics through a dissecting microscope and their positions marked with a marker (Sharpie®). Differentiated regions were removed from HES cell cultures by aspiration with Pasteur pipettes that had been modified to have a 25-200 μm inner tip diameter.

For the self-renewal on topography experiments tissue culture polystyrene was spin coated with a thin layer of Norland Optical Adhesive (NOA), a UV cross-linkable polyurethane. The thin layer of NOA was crosslinked without modification for the flat control surfaces. Patterned surfaces were crosslinked after PDMS (poly(dimethylsiloxane)) molds with the desired topography were used to stamp the pattern into NOA. The elastomer PDMS (Sylgard 184, Dow-Corning, Midland, Mich.) is popular for use in microfluidics and bioMEMS. The patterned and flat NOA surfaces were coated with gelatin at least 24 hours prior to their use for cell culture. Just prior to seeding the MEF feeder cells a coverslip was placed over the topographically patterned area to prevent MEFs from obscuring the pattern. After 24 hours the coverslips were removed, also removing those MEFs that would have landed on the topography. Before the addition of HES cells, the surfaces were rinsed with PBS. HES cells were then plated onto the gelatin coated NOA plates that had MEFs around the perimeter of the plate. The same protocol without the addition of MEFs was used to investigate topography's effect on differentiation.

### Fabrication of Patterned Surfaces

A preferred method of producing the patterned surfaces of the present invention is schematically outlined in FIG. 1. Briefly, soft lithography was used to make cell culture surfaces with nano-scale topography. Silicon wafers were coated in a photoresist that was then patterned with electron beam lithography. The nano-scale areas of silicon exposed by the electron beam were etched with sulfur hexafluoride (SF₆) and tetrafluoroethane (C₂H₂F₄) gases. After the silicon etching was complete, the remainder of the photoresist was removed and the silicon wafer topography was used as a mold for the formation of a PDMS cast. The resulting PDMS cast was used to print nano-scale topography into polyurethane-coated surfaces. The polyurethane surfaces were coated in gelatin, exposed to serum and rinsed with phosphate buffered saline prior to serving as substrates for HES cell culture.

### Cell Behavior

Light microscopy, immunofluorescence microscopy, and scanning electron microscopy were used to observe the surfaces and cell behavior, including cell growth, development, migration, and differentiation.

To determine the rate of cell self-renewal, cell colonies were stained with alkaline phosphatase, a marker of stem cell self-renewal, using the Chemicon® Alkaline Phosphatase Detection Kit for stem cells (Chemicon, Temecula, Calif.), according to the manufacturer's instructions.

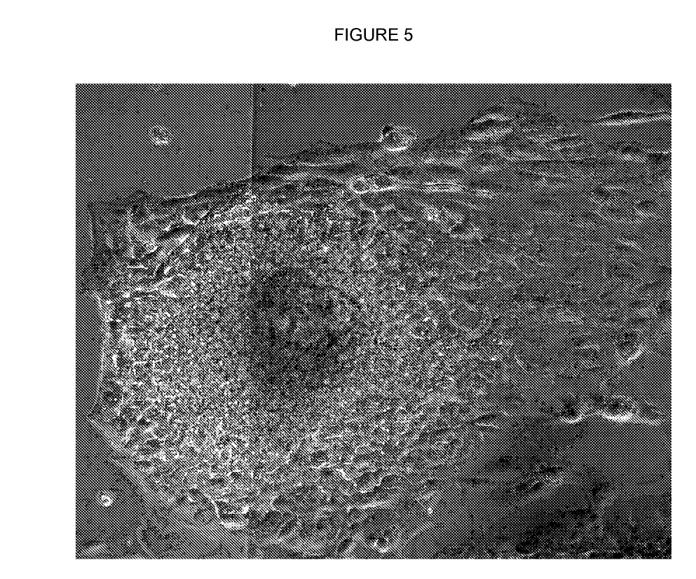

FIG. 5 shows a HES cell colony at the intersection between a flat surface (to the left of the vertical line) and a topographically patterned surface (to the right of the vertical line). Within 48 hours, the presence of 1600 nm pitch topography promoted cell migration along the ridges.

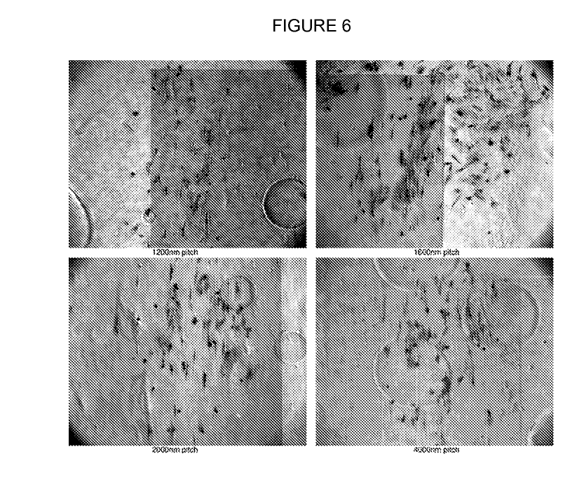

FIG. 6 shows images of human embryonic stem cells that have migrated away from their colonies; many of which have retained high alkaline phosphatase activity, a marker for stem cell pluripotency. Cells were grown for five days on groove and ridge topographic surfaces of different sizes, fixed in 4% paraformaldehyde, and stained with the Chemicon® Alkaline Phosphatase Detection Kit for stem cells (Chemicon, Temecula, Calif.).

As shown in FIG. 6, on topographies at the low end of the micron scale (4 μm, 2 μm, 1.6 μm, and 1.2 μm pitch), morphological changes that indicate differentiation were observed. Many of the stem cells at the perimeter of the colonies were enlarged and spread out on the surface. However, these enlarged, well spread, stem cells retain high alkaline phosphatase activity, a marker of stem cell self-renewal (FIG. 6). These morphologically changed cells would definitely be marked for removal under standard stem cell maintenance protocols on a normal flat surface.

Substrate topography can reduce the frequency of HES cell spontaneous differentiation (p-value<0.0009). Under conditions that encourage self-renewal, a divergent size range of groove and ridge topographies increase the proportion of human embryonic stem cells that stain positive for alkaline phosphatase, a marker of stem cell self-renewal. It is important to note that this is only true when self-renewal promoting factors are present.

In the absence of MEF feeder cells, topography can have a very different effect. In the absence of the self-renewal promoting factors produced by MEF feeder cells, topography actually encourages differentiation. HES cells grown on topography under conditions that lack self-renewal promoters (e.g. without MEF feeder cells) are more prone to the loss of stem cell markers (p=0.019). This emphasizes the importance of the context of the other environmental influences for synergistic effects on the behavior of HES cell cultures.

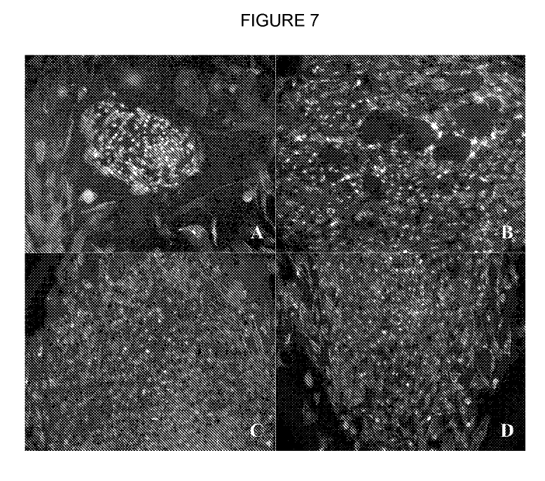

The growth rate of cells on patterned surfaces was examined. Shown in FIG. 7 is the immunofluorescent labeling of Ki67 (a marker of dividing cells).

Cells were cultured on glass coverslips that had been spin coated with NOA then printed with one size topography on each (400 nm, 1400 nm, or 4000 nm pitch) or left flat for use as a control. Cells were rinsed twice with PBS then fixed for 10 min in 4% paraformaldahyde. After two 10 min rinses in PBS, cells were left in blocking solution (5% goat serum, 2% Bovine serum albumin, and 0.1% Triton X-100 in PBS) overnight at 4° C. or for an hour at 37° C. Mouse Anti-Human Ki67 primary antibody (Clone MIB-1, DakoCytomation, Cat# M7240) was used at a 1:200 dilution in blocking solution for one hour at 37° C. After two 10 min rinses in PBS, cells were left in blocking solution for 20-30 min at 37° C. Secondary antibody was goat anti mouse Alexa Fluor® 488 at 1:200 in blocking solution for one hour at 37° C.

Equally high (near 100%) proliferation rates were observed for HES cell colonies on flat and patterned surfaces. No difference was found in HES cell plating efficiency between flat and patterned surfaces. In FIG. 7, Ki67 was labeled with green; actin cytoskeleton was stained using phalloidin and appeared in red, while DAPI staining of the nuclei appeared in blue, as seen in U.S. Provisional Patent Application Ser. No. 60/851,662, which is incorporated herein by reference.

Panel A in FIG. 7 shows a small stem cell colony on a flat surface with most HES cell nuclei staining positive for Ki67 while the surrounding terminally irradiated mouse embryo fibroblasts lack Ki67. Panels B, C, and D in FIG. 7 show stem cell colonies on 400 nm, 1400 nm, and 4000 nm pitch topography respectively, also with most HES cell nuclei staining positive for Ki67.

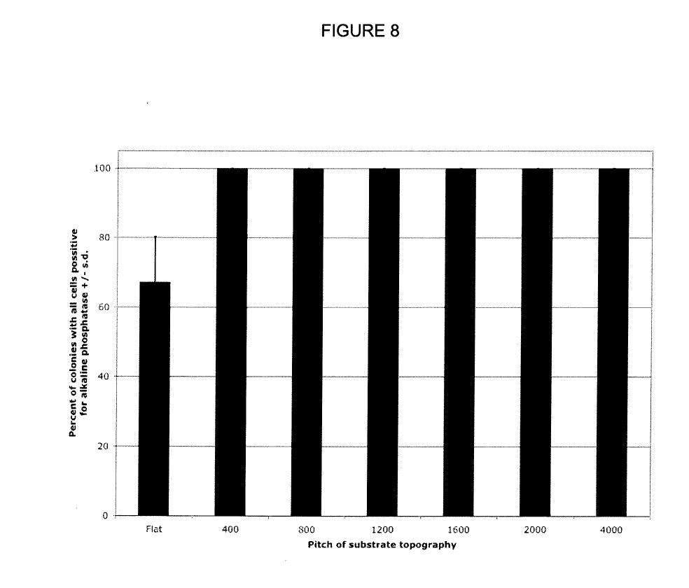

FIG. 8 is a graph showing how nanoscale topography reduces the frequency of spontaneous differentiation in HES cell cultures. Stem cells growth on pitch of substrate topography in the range of 400-4000 nm is shown. The graph depicts the influence of the pitch of the substrate topography on the relative number of colonies stained with alkaline phosphatase, a marker of stem cell self-renewal. After five days on topographically patterned surfaces the frequency of spontaneous differentiation is significantly lower than on flat surfaces (p=0.00092).

### Example 2

### Fabrication of Patterned Surfaces

A range of defined size topographic features was generated utilizing lithographic techniques pioneered for manufacturing computer chips. As shown in one embodiment in FIG. 1, a single patterned substrate can provide a range of feature dimensions. For example, these can range from about 400 nm to about 4000 nm pitch with intervening planar control regions. In a preferred embodiment shown in FIG. 1, the ridge: groove ratio is about 1:1.

The top panel in FIG. 1 shows a simplified schematic of the manufacturing protocol of patterned surfaces. Six steps are shown: coating, X-ray lithography, development, reactive ion etching, cleaning, and low pressure chemical vapor deposition.

The middle left panel shows a chip that has six patterned areas of six different pitches, ranging from 400 nm to 4000 nm. The colors of the patterned areas, which can be seen in U.S. Provisional Patent Application Ser. No. 60/851,662, incorporated herein by reference, are from diffraction caused by the grooves and ridges. Pitch is defined as the distance from the start of one ridge to the start of the next ridge.

The middle right panel in FIG. 1 is an electron microscope image of the edge of a 400 nm pitch patterned area. The silicon wafer was used as a mold for the formation of a PDMS cast. The resulting PDMS cast was used to print nano-scale topography into polyurethane-coated surfaces. The polyurethane surfaces were coated in gelatin, exposed to mouse embryo fibroblast media, and rinsed with phosphate buffered saline prior to serving as substrates for HES cell culture.

As shown in FIG. 1 (bottom panels), atomic force microscopy was used to confirm that the addition of gelatin and serum did not obscure substrate topography.

### Example 3

### Migration

As shown in FIG. 5, topographic cues promote migration with retention of self-renewal properties. A HES cell colony was plated at the intersection between a flat surface (to the left of the vertical line, i.e. intersection) and a topographically patterned surface (to the right of the vertical line, ie. intersection). Within 48 hours, the presence of 1600 nm pitch topography promoted cell migration along the ridges.

### Example 4

### Proliferation

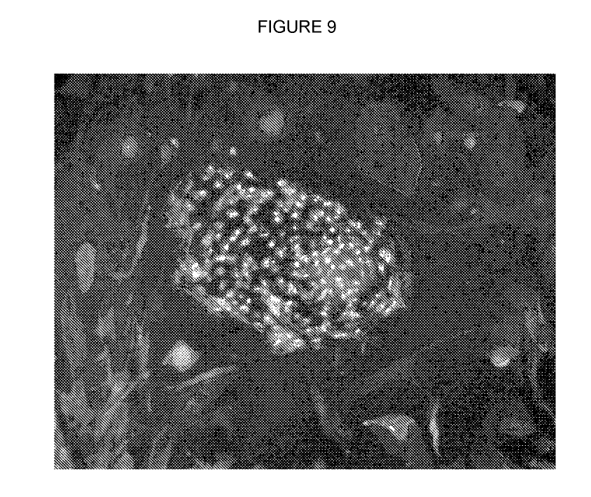

As shown in FIG. 9, proliferation was unaffected by the presence or scale of topographic cues (unlike other differentiated cell types investigated).

### Example 5

### Differentiation

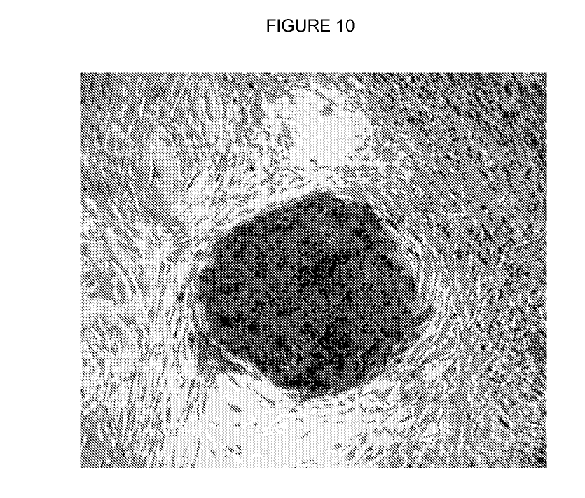

As shown in FIG. 10, topographic cues strongly promote stem cell self-renewal (independent of scale). FIG. 10 depicts an alkaline phosphatase-stained HES cell colony. Alkaline phosphatase, a known marker of pluripotent stem cells, was used to distinguish differentiated colonies from those that retain pluripotent potential. There was approximately 100% self-renewal at 5 days.

Cell culture substrates with nanoscale topography increase the likelihood of embryonic stem cell self-renewal under stem cell propagation conditions. This has broad application to stem cell biology, as the use of topographic cueing (using specific dimensional features) can participate in determining the differentiation fate of embryonic stem cells and participate with other soluble factors in ultimately determining the pathways of differentiation pursued (or the retention of the de-differentiated state).

All publications, patents, and patent applications cited herein are hereby incorporated by reference in their entirety for all purposes to the same extent as if each individual publication, patent, or patent application were specifically and individually indicated to be so incorporated by reference. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of the disclosure.

What is claimed is:

1. A method for growing cells in vitro which comprises (a) contacting a suspension of cells in a cell culture medium with a patterned surface for growth of cells comprising a planar surface having nanotextured topography with longitudinal grooves and projections extending perpendicularly to the planar surface, and (b) incubating the cells under cell growth or differentiation conditions to maintain the cells in their desired growth and differentiation state.

2. The method of claim 1 wherein the cells are stem cells.

3. The method of claim 1 wherein the cells are embryonic stem cells.

4. The method of claim 1 wherein the cells are human embryonic stem cells.

5. The method of claim 1 wherein the longitudinal grooves and projections have a pitch size of between about 40 nm and about 8000 nm.

6. The method of claim 1 wherein the size ratio of a projection to a longitudinal groove is approximately 1:1.

7. The method of claim 1 wherein, under culture conditions that promote cell differentiation, the topography of the surface promotes cell differentiation as compared to cell differentiation on a surface in the absence of the topography.

8. The method of claim 1 wherein, under culture conditions that promote cell self-renewal, the topography of the surface promotes cell self-renewal as compared to cell self-renewal on a surface in the absence of the topography.

9. The method of claim 1, wherein the planar surface is formed from a material with functional groups capable of forming a covalent bond with a biomolecule selected from the group consisting of a peptide, a protein, a polynucleotide, a polysaccharide, a lipid, a growth factor, and a bioactive agent.

10. A kit comprising: (a) a patterned surface for growth of cells comprising a planar surface having nanotextured topography with longitudinal grooves and projections extending perpendicularly to the planar surface, and (b) an undifferentiated cell.

11. The kit of claim 10 wherein the undifferentiated cell is a stem cell.

12. The kit of claim 10 wherein the undifferentiated cell is an embryonic stem cell.

13. The kit of claim 10 wherein the undifferentiated cell is a human embryonic stem cell.

14. The kit of claim 10 wherein the longitudinal grooves and projections have a pitch size of between about 40 nm and about 8000 nm.

15. The kit of claim 10 wherein the size ratio of a projection to a longitudinal groove is approximately 1:1.

16. The kit of claim 10 further comprising culture medium.

17. The kit of claim 10 wherein, under culture conditions that promote cell differentiation, the topography of the surface promotes cell differentiation as compared to cell differentiation on a surface in the absence of the topography.

18. The kit of claim 10 wherein, under culture conditions that promote cell self-renewal, the topography of the surface promotes cell self-renewal as compared to cell self-renewal on a surface in the absence of the topography.

19. The kit of claim 10, wherein the planar surface is formed from a material with functional groups capable of forming a covalent bond with a biomolecule selected from the group consisting of a peptide, a protein, a polynucleotide, a polysaccharide, a lipid, a growth factor, and a bioactive agent.
